(19) United States
(12) Patent Application Publication
(10) Pub. No.: US 2025/0057441 A1
(43) Pub. Date: Feb. 20, 2025

(54) IMPLEMENTATION OF ADVANCED MEMS/NEMS BIOSENSORS FOR HUMAN BREATH AND BODY GAS ANALYSES

(71) Applicant: HONDA MOTOR CO., LTD., Tokyo (JP)

(72) Inventors: Dong ZHAO, San Jose, CA (US); Gugang CHEN, Palo Alto, CA (US)

(21) Appl. No.: 18/450,136

(22) Filed: Aug. 15, 2023

(51) Int. Cl. A61B 5/08; A61B 5/00; A63F 13/40

(52) U.S. Cl. CPC A61B 5/082; A61B 5/0002; A61B 5/6803; A63F 13/40; A61B 2562/028

(57) ABSTRACT

A system for collecting bioinformatic data includes an array of micro/nano electro-mechanical system (MEMS/NEMS) sensors arranged on an article such that the array of MEMS/NEMS sensors are positioned within an acceptable range of a gas source of a subject. Each MEMS/NEMS sensor is configured to generate a signal based on a concentration of one or more gaseous biomarkers within a gas emitted from the subject. The system includes a preprocessor coupled with the array of MEMS/NEMS sensors and configured to generate one or more indicators based on the signal from each of a plurality of the MEMS/NEMS sensors. The system includes a computing device comprising one or more memories storing computer-executable instructions and one or more processors, individually or in combination, configured to execute the computer-executable instructions to cause the computing device to apply the one or more indicators to a model to output a health status of the subject.

### TECHNICAL FIELD

The present disclosure is directed to devices and systems having one or more biosensors for collecting bioinformatic data.

### BACKGROUND

Gases released from mammals, particularly humans, via the breath and skin provide a wealth of information regarding an individual's health status. As such, identifying and analyzing these gases would provide valuable, non-invasive insights into a human subject's health. However, the deployment of sensor units for such an analysis presents a significant challenge due to the low concentration of biomarkers in a human's gaseous secretions, as this presents the risk of the gases being unacceptably diluted and/or contaminated prior to analysis.

### SUMMARY

The present disclosure is directed to a system for collecting bioinformatic data, the system having an array of micro/nano electro-mechanical system (MEMS/NEMS) sensors arranged on an article such that the array of MEMS/NEMS sensors are positioned within an acceptable range of a gas source of a subject, each MEMS/NEMS sensor configured to generate a signal based on a concentration of one or more gaseous biomarkers within a gas emitted from the subject; a preprocessor coupled with the array of MEMS/NEMS sensors and configured to generate one or more indicators based on the signal from each of a plurality of the MEMS/NEMS sensors; a computing device having one or more memories storing computer-executable instructions and one or more processors, individually or in combination, configured to execute the computer-executable instructions to cause the computing device to apply the one or more indicators to a model to output a health status of the subject.

### BRIEF DESCRIPTION OF THE DRAWINGS

FIG. 1 shows a schematic view of an example operating environment of a sensor device.

FIG. 2A shows an example device according to aspects of the present disclosure.

FIG. 2B shows an example device according to aspects of the present disclosure.

FIG. 3 shows an example system according to aspects of the present disclosure.

FIG. 4 shows an example device according to aspects of the present disclosure.

FIG. 5 shows an example device according to aspects of the present disclosure.

FIG. 6A shows an example device according to aspects of the present disclosure.

FIG. 6B shows an example device according to aspects of the present disclosure.

FIG. 6C shows an example device according to aspects of the present disclosure.

FIG. 6D shows an example device according to aspects of the present disclosure.

FIG. 6E shows an example device according to aspects of the present disclosure.

FIG. 6F shows an example device according to aspects of the present disclosure.

FIG. 6G shows an example device according to aspects of the present disclosure.

FIG. 6H shows an example device according to aspects of the present disclosure.

FIG. 6I shows an example device according to aspects of the present disclosure.

FIG. 7A shows an example device according to aspects of the present disclosure.

FIG. 7B shows an example device according to aspects of the present disclosure.

FIG. 7C shows an example device according to aspects of the present disclosure.

### DETAILED DESCRIPTION

The present disclosure is directed to sensor units, devices, and systems configured to collect and/or provide bioinformatic data. According to some aspects, the device may include one or more biosensors positioned relative to a user sufficient for collecting reliable bioinformatic data. In some examples, the one or more biosensors are based on microelectromechanical systems (MEMS) and/or nanoelectromechanical systems (NEMS).

As used herein, a “microelectromechanical system” or “MEMS” refers to a microscopic device having both electronic and mechanical components that function on the microscale. For example, a MEMS may include one or more microsensors configured to collect information from an environment and which are in communication with a data-processing unit. As used herein, a “nanoelectromechanical system” or “NEMS” refers to a device having both electronic and mechanical components that function on the nanoscale. For example, a NEMS may include one or more nanosensors configured to collect information from an environment and which are in communication with a data-processing unit.

It should be understood that a biosensor as described herein refers to a functional self-contained unit. In some non-limiting examples, the biosensor may include one or more sensors for collecting data from an external environment, a power supply, and/or one or more data transmission component(s), such as RF component(s). The biosensor may be provided as part of a sensor unit, which may include the biosensor in addition to other functional components designed for data processing, storage, and/or display.

According to some aspects, the device of the present disclosure may include one or more biosensors positioned such that when the device is used by a user, the one or more biosensors are within an acceptable range of the user. In some non-limiting examples, the device may be wearable. Examples of wearable devices include, but are not limited to, headsets, helmets, hats, masks, gloves, shoes, socks, belts, belt buckles, shirts, ties, buttons, undergarments, outerwear (e.g., coats, scarves, earmuffs), pants, jewelry (e.g., rings, necklaces, earrings, watches), components thereof, and combinations thereof. However, the present disclosure is not limited to wearable devices. According to some aspects, the device may include any device that is usable within an acceptable range of a user. Non-limiting examples include phones, handles (e.g., door handles, cabinet handles, appliance handles, automobile handles), tools (e.g., hammers, wrenches, pliers), cutlery (e.g., forks, spoons, knives, chopsticks), keys, key chains, remote controls, automobile components (e.g., steering wheels, seats), writing utensils (e.g., pens, pencils), computer components (e.g., mice, keyboards), food and drink containers, instruments, microphones, speakers, tables, toys, and combinations thereof.

As used herein, an “acceptable range” refers to a distance from a user sufficient for a biosensor to collect reliable bioinformatic data from the user. In one example, bioinformatic data may be provided by a user's breath. In this example, an acceptable range may refer to a distance from a user's mouth and/or nose sufficient for a biosensor to collect reliable bioinformatic data from the user's breath. Additionally or alternatively, bioinformatic data may be provided by a user's skin. In this example, an acceptable range may refer to a distance from a user's skin sufficient for a biosensor to collect reliable bioinformatic data from the user's skin. Non-limiting examples of acceptable ranges include no more than about 30 cm, optionally no more than about 25 cm, optionally no more than about 20 cm, optionally no more than about 15 cm, optionally no more than about 10 cm, optionally no more than about 5 cm, optionally no more than about 4 cm, optionally no more than about 3 cm, optionally no more than about 2 cm, and optionally no more than about 1 cm. In some non-limiting examples, there may be no distance between a user and the one or more biosensors, for example, wherein the one or more biosensors are in direct contact with a user. In some implementations, the acceptable range when the bioinformatic data is provided by a user's breath may be less than the acceptable range when the bioinformatic data is provided by gas emitted from a user's skin.

According to some aspects, the bioinformatic data may include the composition of a user's breath, the composition of gas secretions from a user's skin, or a combination thereof. In some examples, the bioinformatic data may include one or more gaseous biomarkers. As used herein, a “gaseous biomarker” refers to a gas species that is detectable by a biosensor as disclosed herein. It should be understood that a gaseous biomarker may be a component of a user's breath and/or a component of a user's skin secretions. Non-limiting examples of gaseous biomarkers include nitric oxide, ammonia, methanol, hydrogen, hydrogen sulfide, nitrogen dioxide, acetone, isoprene, carbon monoxide, carbon dioxide, methane, formaldehyde, dimethyl sulfide, acetaldehyde, methanol, butadiene, methanethiol, ethyl acetate, butyl acetate, styrene, 2-methyl heptane, 2,2,4,6,6-pentamethyl heptane, 1-heptene, decane, undecane, propyl benzene, methyl cyclopropane, 1-methyl-2-pentyl cyclopropane, trichlorofluoromethane, benzene, 1,2,4-trimethyl benzene, 3-methyl octane, hexane, heptane, 1,4-dimethyl benzene, 2,4-dimethyl heptane, cyclohexane, and combinations thereof.

According to some aspects, the device of the present disclosure may include one or more biosensors positioned such that when the device is used by a user, the one or more biosensors are within an acceptable range of the user such that a concentration of gaseous biomarkers provided to the one or more biosensors from the user is between about 0.1 and 2000 parts per billion (ppb). It should be understood that the acceptable range may depend, at least in part, on the identity of the gaseous biomarker. For example, Table 1 shows example concentration ranges for several example gaseous biomarkers.

TABLE 1

| | Example Acceptable |
|---|---|
| Gaseous | Concentration Range |
| Biomarker | (ppb) |
| nitric oxide | 6-30 |
| acetone | 1.2-1880 |
| isoprene | 12-580 |
| butadiene | 0.2-10 |
| methanol | 160-2000 |
| hydrogen sulfide | 50-110 |
| ethyl acetate | 0.14-10 |
| butyl acetate | 0.6-20 |

According to some aspects, the concentration of gaseous biomarkers provided to the one or more biosensors may be sufficient to detect an increased risk of a condition correlated with the gaseous biomarker(s). For example, the concentration of nitric oxide provided to the one or more biosensors by a user may be sufficient to detect an increased risk of asthma. In another example, the concentration of acetone provided to the one or more biosensors by a user may be sufficient to detect an increased risk of unhealthy glucose levels in a patient suffering from diabetes.

Additionally or alternatively, the concentration of gaseous biomarkers provided to the one or more biosensors may be sufficient to detect a cognitive state or change thereof correlated with the gaseous biomarker(s). In one non-limiting example, the concentration of nitric oxide and/or methanol provided to the one or more biosensors by a user may be sufficient to detect an improvement in mental state (e.g., an improved mood). In another example, the concentration of alcohol provided to the one or more biosensors by a user may be sufficient to detect an unacceptable blood alcohol content.

According to some aspects, the devices and/or systems of the present disclosure may include one or more flow sensors, such as a flow sensor configured to measure the flow rate of a user's breath. In some non-limiting examples, the one or more flow sensors are based on MEMS and/or NEMS as described herein.

According to some aspects, the device of the present disclosure may include one or more flow sensors positioned such that when the device is used by a user, the one or more flow sensors are within an acceptable range of the user sufficient to determine a flow rate of gas provided to the one or more flow sensors from the user. In this way, the one or more flow sensors may detect a change in flow rate of gas (e.g., from a user's breath), which may correspond with a certain condition and/or cognitive state or change thereof. In one non-limiting example, the one or more flow sensors may detect an increase in flow rate of a user's breath, which may correspond with an improvement of a user's cognitive state (e.g., an improvement in mood).

In some non-limiting examples, the device may include one or more pumps configured to direct gas from a user to the one or more biosensors. According to some aspects, the one or more pumps may be selectively activatable and/or adjustable such that the flow rate of the gas directed to the one or more biosensors is selectable.

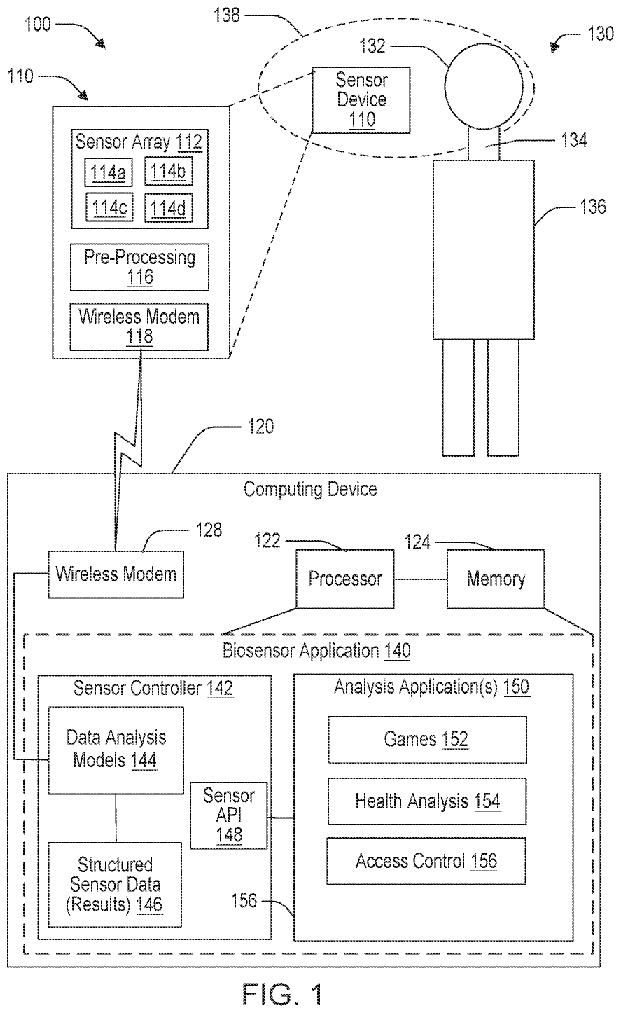

Turning to FIG. 1, a schematic view of an example operating environment 100 of a sensor device 110 and example methods according to an aspect of the disclosure are provided. The sensor device 110 may be configured to determine biomarkers corresponding to one or more subjects 130.

As illustrated, the sensor device 110 includes a sensor array 112 including one or more sensors (e.g., sensors 114a-114d). Each of the sensors 114 may be, for example, a MEMS or NEMS configured to generate a signal indicative of a measured quantity.

The sensor device 110 may further include pre-processing block 116 configured to perform pre-processing of the signals from the sensors 114. For example, the pre-processing block 116 may digitize, quantize, and/or summarize the signals. The pre-processing block 116 may be configured, for example, as a circuit, field-programmable gate array (FPGA), or digital signal processor. The pre-processing block 116 may include or be connected to a memory for storing pre-processed data.

The sensor device 110 may include a wireless modem 118. The wireless modem 118 may be configured to implement a wireless communications protocol for communicating with a computing device 120. For example, the wireless communications protocol may be a short range wireless communications protocol such as Bluetooth, Bluetooth Low Energy, Zigbee, Wi-Fi, etc. The wireless modem 118 may transmit the pre-processed data from the sensor device 110 to the computing device 120.

The subject 130 may be a human that interacts with the sensor device 110. From the perspective of the sensor device, the human 130 may be considered to include a face 132, voice box 134, and body 136. As discussed in further detail below, the sensor device may detect gaseous biomarkers emitted from the human 130. In some implementations, the sensor device 110 may be associated with acceptable range 138 of the human 130.

The computing device 120 may be configured to receive pre-processed biomarker data from the sensor device 110, perform analysis of the biomarker data, and use analyzed biomarker data as input into an application. In an aspect, the computing device includes a processor 122, a memory 124, and a wireless modem 128.

The processor 122 may include one or more processors for executing instructions. An example of processor 122 can include, but is not limited to, any processor specially programmed as described herein, including a controller, microcontroller, application specific integrated circuit (ASIC), field programmable gate array (FPGA), system on chip (SoC), or other programmable logic or state machine. The processor 122 may include other processing components such as an arithmetic logic unit (ALU), registers, and a control unit. The processor 122 may include multiple cores and may be able to process different sets of instructions and/or data concurrently using the multiple cores to execute multiple threads.

Memory 124 may be configured for storing data and/or computer-executable instructions defining and/or associated with an operating system and/or application, and processor 122 may execute operating system 452 and/or applications (e.g., biosensor application 140). Memory 124 may represent one or more hardware memory devices accessible to computing device 120. An example of memory 124 can include, but is not limited to, a type of memory usable by a computer, such as random access memory (RAM), read only memory (ROM), tapes, magnetic discs, optical discs, volatile memory, non-volatile memory, and any combination thereof. Memory 124 may store local versions of applications being executed by processor 122. In an implementation, the memory 124 may include a storage device, which may be a non-volatile memory.

The wireless modem 118 may be configured to perform a wireless communication protocol for communicating with the sensor device 110. For example, the wireless communications protocol may be a short range wireless communications protocol such as Bluetooth, Bluetooth Low Energy, Zigbee, Wi-Fi, etc.

The processor 122 and the memory 124 may be configured to execute a biosensor application 140. The biosensor application 140 may include a sensor controller 142 and analysis applications 150.

The sensor controller 142 may be configured to receive and analyze the pre-processed biomarker data from the sensor device 110 via the wireless modems 118 and 128. The sensor controller 142 may include one or more data analysis models 144. The data analysis models 144 may be configured to determine various results 146 from the pre-processed biomarker data. For instance, the data analysis models may analyze the pre-processed biomarker data to output a concentration of one or more gases. In some implementations, the data analysis models may analyze the concentrations to output a health condition such as a stress level. The sensor controller 142 may include a sensor application interface (API) 148 configured to allow analysis applications 150 to request or subscribe to sensor data 146.

The analysis applications 150 may include one or more software applications that utilize sensor data 146. For example, the analysis applications 150 may include games 152, health analysis 154, or access control 156, as described herein.

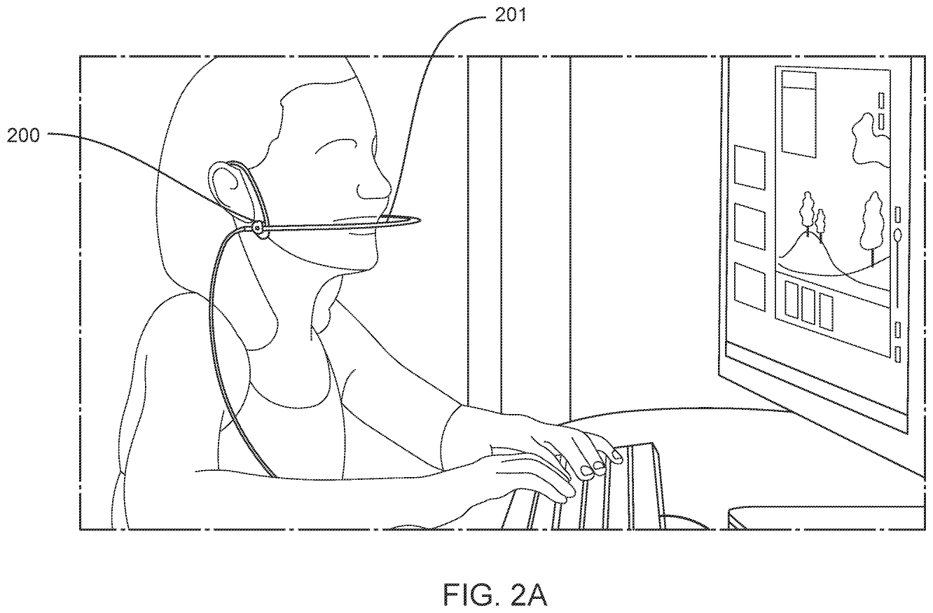

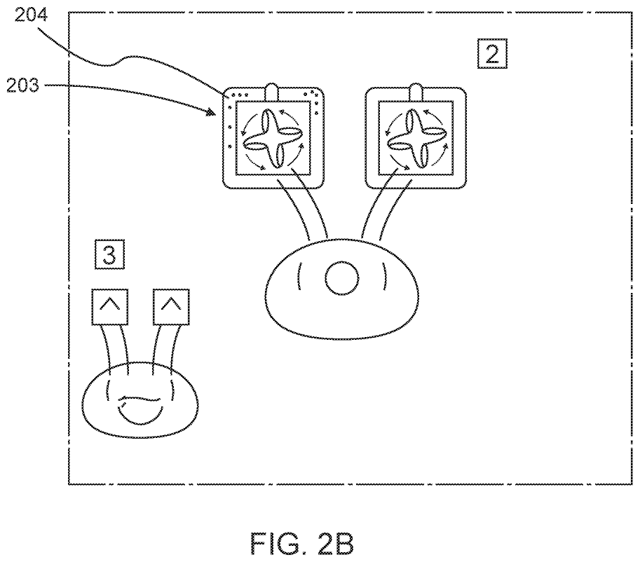

FIG. 2A shows one example device according to aspects of the present disclosure. In particular, FIG. 2A shows a headset 200 wearable by a user. Headset 200 may include one or more biosensors 201 configured to be positioned within an acceptable range of a user's mouth when worn such that biosensors 201 may collect gaseous biomarkers from the user. Headset 200 may be in communication with a computing device.

In an implementation, for example, the computing device includes one or more computer memories storing computer-executable instructions (e.g., a computer program). The computing device includes one or more processors, individually or in combination, configured to execute the computer-executable instructions. In some implementations, the computer-executable instructions may include an application that uses signals from the biosensors 101 as input. For example, the application may include the games 152. The games 152 may encourage the subject to provide the gas source (e.g., breath) to perform a task in the game. The games 152 may encourage consistent and/or repeatable input for monitoring a health condition of the user.

In this way, headset 200 may be provided as part of a system configured to detect and/or measure gaseous biomarkers from a user sufficient to detect an increased risk of a condition and/or a change in cognitive state as described herein.

FIG. 2B shows another example device according to aspects of the present disclosure. In particular, FIG. 2B shows a pinwheel 203 having one or more apertures 204. Each one of apertures 204 may be in communication with one or more biosensors and/or one or more flow sensors as described herein. In this example, pinwheel 203 may be configured such that when it is used by a user, one or more apertures 204 are within an acceptable range of a user such that when the user exhales onto pinwheel 203, the one or more biosensors and/or one or more flow sensors may collect reliable bioinformatic data as described herein. In this way, pinwheel 203 may be provided as part of a system configured to detect and/or measure gaseous biomarkers and/or other bioinformatic data from a user sufficient to detect an increased risk of a condition and/or a change in cognitive state as described herein.

The present disclosure is also directed to systems configured to be usable by two or more users (e.g., a group of subjects) simultaneously. For example, the system may include two or more discrete biosensors and/or two or more discrete flow sensors configured to collect reliable bioinformatic data from each user as described herein. In another example, the system may include a single biosensor and/or a single flow sensor configured to collect reliable bioinformatic data from each user simultaneously.

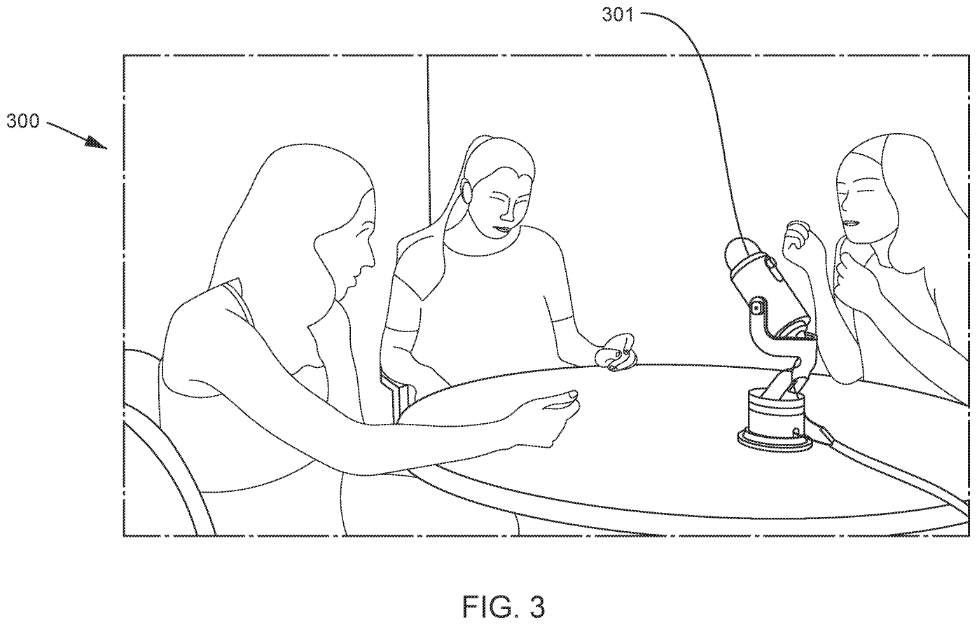

FIG. 3 shows an example of a system according to aspects of the present disclosure. As shown in FIG. 3, system 300 may include a microphone 301 having one or more biosensors and/or one or more flow sensors as described herein. In this way, microphone 301 may collect reliable bioinformatic data from each user during conversation between users as described herein. According to some aspects, the data may be compiled to provide collective bioinformatic data, that is, bioinformatic data on a group of individuals. For example, the bioinformatic data may represent a stress level of the group of individuals.

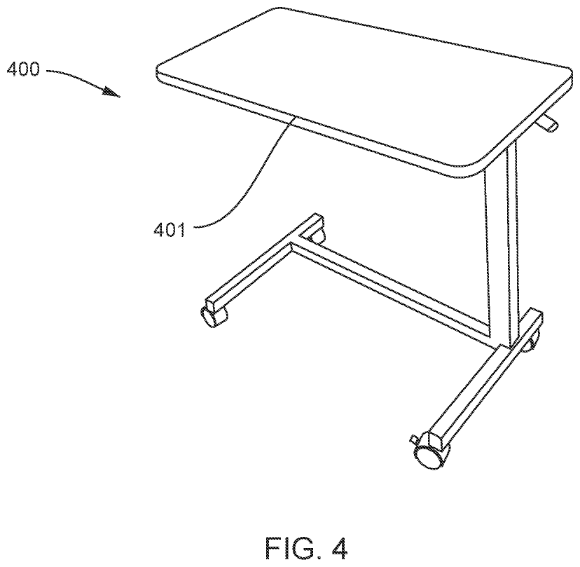

FIG. 4 shows another example device according to the present disclosure. As shown in FIG. 4, the device may include a table 400 having one or more biosensors and one or more flow sensors 401 provided thereon. For instance, the sensors 401 may be located on an edge of the table 400 facing the subject. The sensors 401 may be positioned within an acceptable range of the wrists or hands of the user (e.g., when typing on a keyboard) and/or within an acceptable range of the mouth and/or nose of the user.

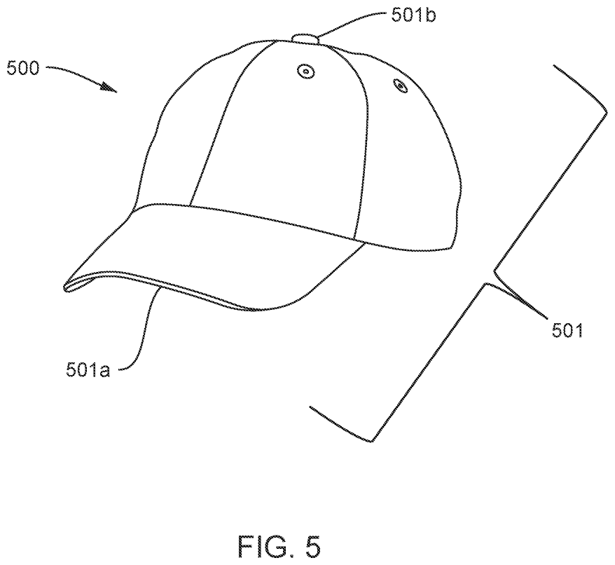

FIG. 5 shows another example device according to the present disclosure. As shown in FIG. 5, the device may include a hat 500 having one or more biosensors 501. In particular, one or more biosensors 501 may include a first portion 501a, such as a mouthpiece, configured to collect reliable bioinformatic data from the breath of a wearer as described herein. One or more biosensors 501 may also include a second portion 501b in communication with first portion 501a. In this example, second portion 501b may include power supply and data transmission component(s), as described herein. As shown in FIG. 5, second portion 501b may be provided a certain distance from first portion 501a.

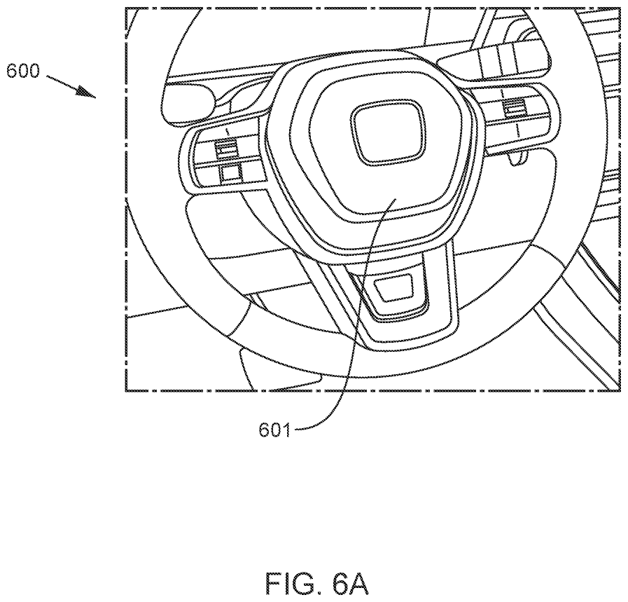

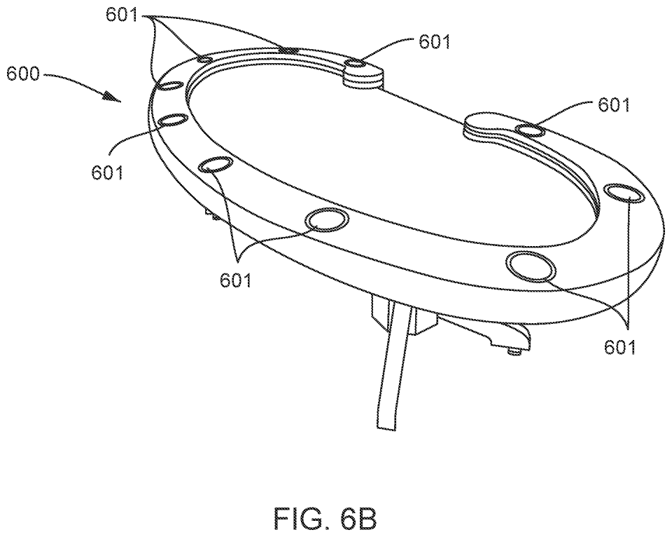

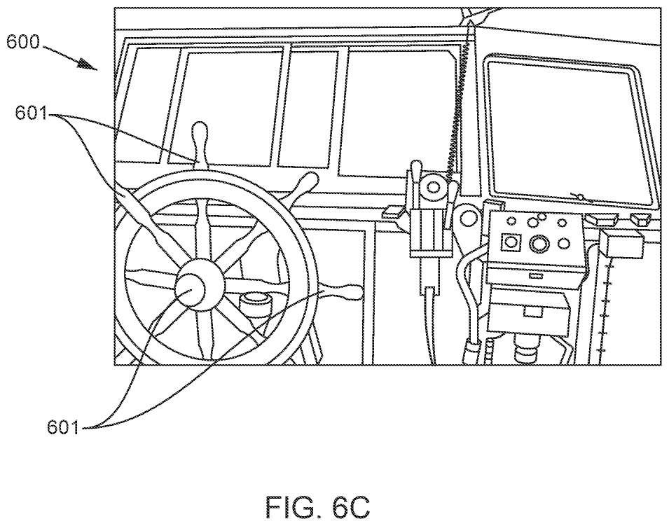

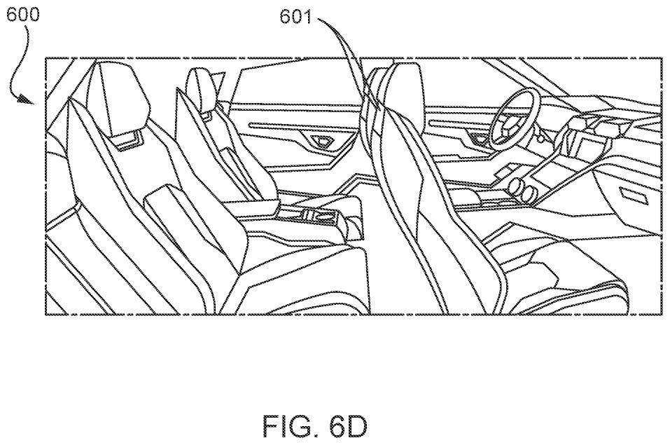

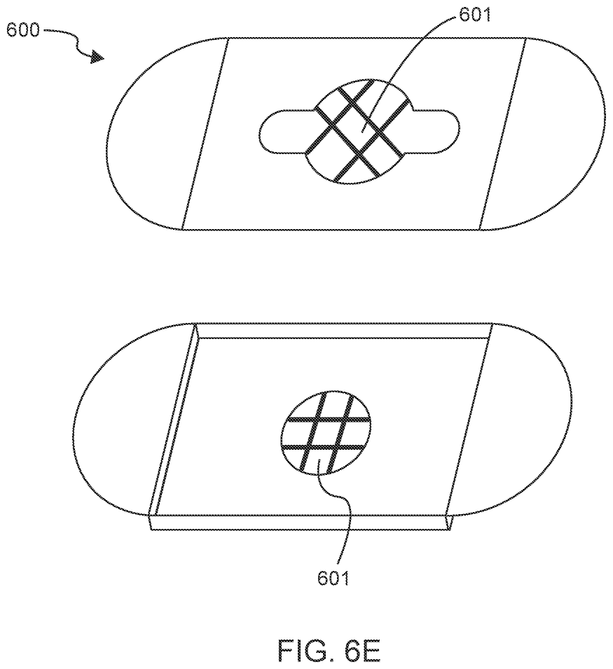

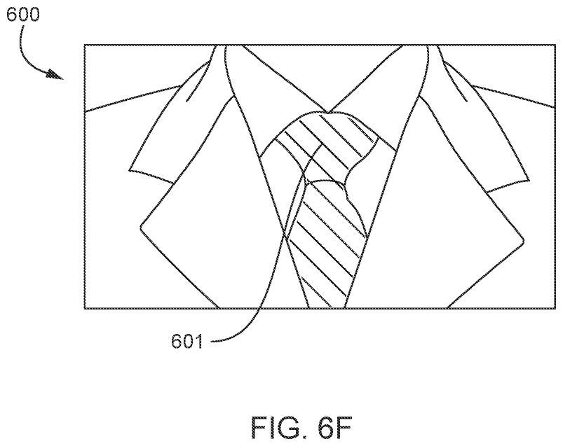

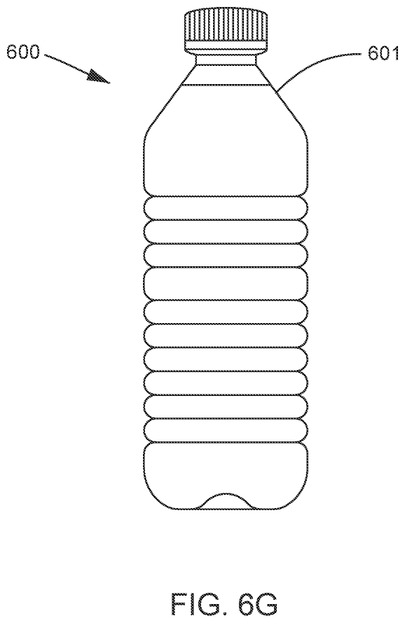

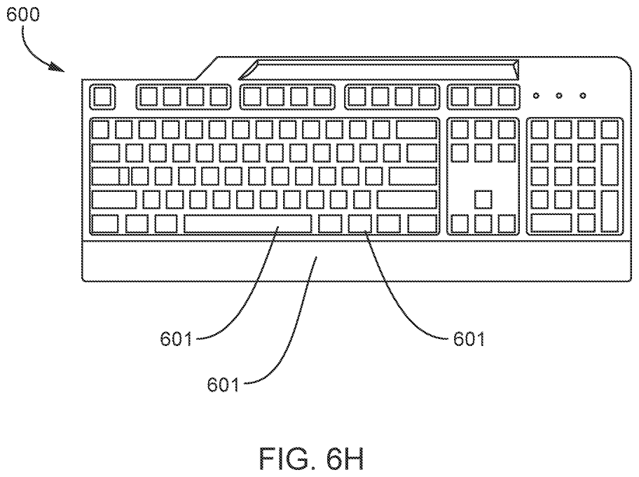

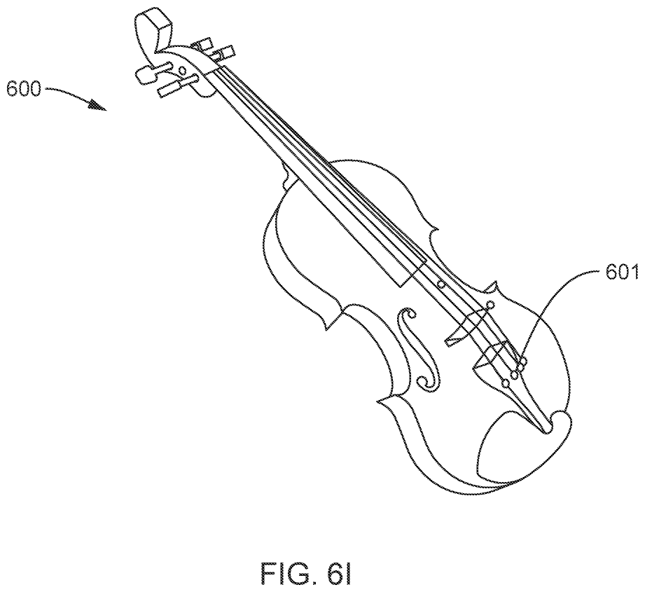

FIGS. 6A-6I each show example devices 600 having one or more biosensors 601 configured to collect reliable bioinformatic data from a user's breath during use as described herein. For instance, in FIG. 6A, a sensor 601 may be located in a steering wheel of a vehicle. In some implementations, the system 100 may control access to a vehicle, for example, to prevent ignition, impose a maximum speed, or deploy relaxation features (e.g., music, lighting, or massage) in response to a detected health status of the user. In another example, in FIG. 6D, the sensors may be located in a headrest of the vehicle. As another example, in FIG. 6C, the sensors 601 may be located on the wheel of a vessel to control access to similar features of the vessel. As another example, in FIG. 6E, the sensors 601 may be located on a mask. In some implementations, the system 100 may detect a respiratory status of the subject wearing the mask. In another example, in FIG. 6F, the sensors 601 may be located in a neck tie positioned near the mouth of the subject. In FIG. 6G, a sensor 601 may be located near the mouth of a drinking container (e.g., a bottle or cup). In yet another example, in FIG. 6I, the sensor 601 may be located near the chin rest of an instrument to collect samples from the breath of the subject.

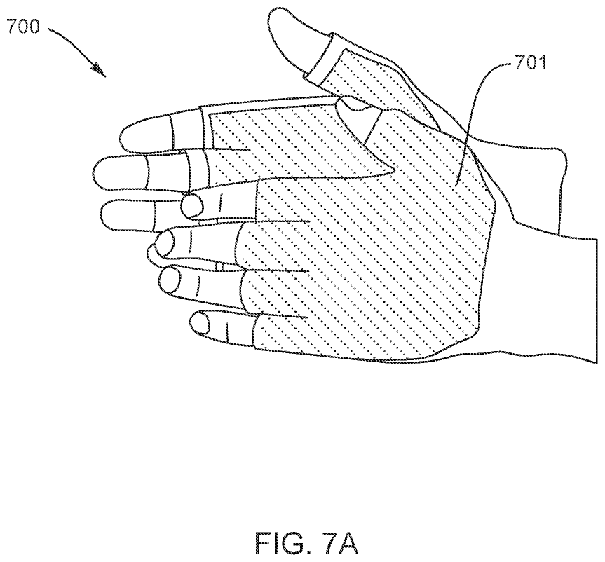

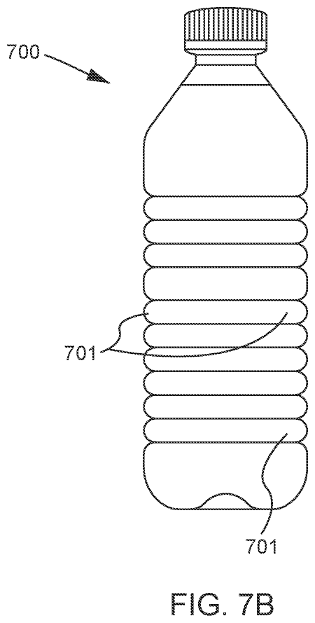

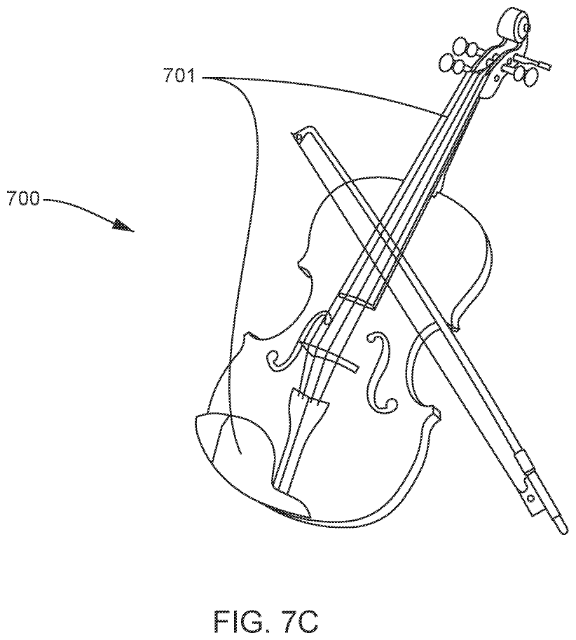

According to some aspects, the location of a biosensor may be selected based on the desired bioinformatic data to be collected. For example, in the case wherein the desired bioinformatic data include ammonia, the device may include one or more biosensors configured to be proximal a user's hand when used, as ammonia secretion is most concentrated in a human's hand. FIGS. 7A-7C show example devices adapted for collecting such bioinformatic data. In particular, FIGS. 7A-7C show devices 700 having one or more biosensors 701 that are located such that when a user is using devices 700, one or more biosensors 701 are proximal the user's hand(s). For example, in FIG. 7A, device 700 is one or more gloves. In this example, one or more biosensors 701 are located on finger portions and/or on a palmar and/or dorsal side of the glove such that the one or more biosensors 701 are in contact with a subject's hand when worn. In another example, in FIG. 7B, the sensors 701 may be located on the sides of the drinking container to collect gas from the skin of the subject, in contrast to the sensor 601 located near the mouth of the drinking container. Similarly, in FIG. 7C, the sensors 701 may be located on the neck or bow of the instrument to collect samples from gas emitted by the skin of the subject.

What is claimed is:

1. A system for collecting bioinformatic data, comprising:
an array of micro/nano electro-mechanical system (MEMS/NEMS) sensors arranged on an article such that the array of MEMS/NEMS sensors are positioned within an acceptable range of a gas source of a subject, each MEMS/NEMS sensor configured to generate a signal based on a concentration of one or more gaseous biomarkers within a gas emitted from the subject;
a preprocessor coupled with the array of MEMS/NEMS sensors and configured to generate one or more indicators based on the signal from each of a plurality of the MEMS/NEMS sensors;
a computing device comprising one or more memories storing computer-executable instructions and one or more processors, individually or in combination, configured to:
execute the computer-executable instructions to cause the computing device to apply the one or more indicators to a model to output a health status of the subject.

2. The system of claim 1, wherein the one or more processors, individually or in combination, are configured to cause the computing device to:
prompt the subject to provide a breath input to the array of MEMS/NEMS sensors; and
control a game based on the breath input, the one or more biomarkers, or the health status.

3. The system of claim 1, wherein the one or more processors, individually or in combination, are configured to cause the computing device to:
detect a medical condition of the subject based on the health status.

4. The system of claim 1, wherein the one or more processors, individually or in combination, are configured to cause the computing device to:
control access to a feature of the article or the computing device based on the health status.

5. The system of claim 1, wherein the gas source is a mouth or nose of the subject, and wherein the article is a microphone located on a robotic arm configured to move the microphone and array of MEMS/NEMS sensors within the acceptable range.

6. The system of claim 1, wherein the subject is a group of human subjects and the health status is applicable to the group.

7. The system of claim 1, wherein the gas source is skin of the subject.

8. The system of claim 6, wherein the article is an article of clothing.

9. The system of claim 7, wherein the acceptable range is no more than about 5 cm.

10. The system of claim 1, wherein the gas source is a mouth or nose of the subject.

11. The system of claim 9, wherein the article is selected from a headset, a helmet, a hat, and a mask.

12. The system of claim 11, wherein the acceptable range is no more than about 30 cm.

13. The system of claim 1, further comprising:
a first wireless modem located on the article and coupled with the preprocessor and configured to transmit the one or more indicators; and
a second wireless modem coupled to the one or more processors and configured to receive the one or more indicators.

14. The system of claim 1, wherein the health status is one of: a stress level, an anxiety level, inflammation level, glucose level, or toxicity level.

15. The system of claim 1, wherein the subject is a human.

16. A method of collecting bioinformatic data, comprising:
positioning an array of micro/nano electro-mechanical system (MEMS/NEMS) sensors arranged on an article within an acceptable range of a gas source of a subject, each MEMS/NEMS sensor configured to generate a signal based on a concentration of one or more gaseous biomarkers within a gas emitted from the subject;
generating one or more indicators based on the signal from each of a plurality of the MEMS/NEMS sensors;
applying the one or more indicators to a model to output a health status of the subject.

17. The method of claim 16, further comprising:
prompting the subject to provide a breath input to the array of MEMS/NEMS sensors; and
controlling a game based on the breath input, the one or more biomarkers, or the health status.

18. The method of claim 16, further comprising detecting a medical condition of the subject based on the health status.

19. The method of claim 16, further comprising controlling access to a feature of the article or the computing device based on the health status.

20. The method of claim 16, wherein the article is a microphone located on a robotic arm, further comprising moving the microphone and array of MEMS/NEMS sensors within the acceptable range.
